(19) United States
(12) Patent Application Publication
(10) Pub. No.: US 2014/0213882 A1
(43) Pub. Date: Jul. 31, 2014

(54) ELECTRODE STRUCTURE FOR MEASURING BIO-SIGNAL AND APPARATUS FOR MEASURING ELECTROCARDIOGRAM USING THE SAME

(71) Applicant: Pukyong National University Industry-University Cooperation Foundation, (US)

(72) Inventor: Wan Young CHUNG, Busan (KR)

(73) Assignee: Pukyong National University Industry-University Cooperation Foundation, Busan (KR)

(21) Appl. No.: 13/851,437

(22) Filed: Mar. 27, 2013

(30) Foreign Application Priority Data
Jan. 31, 2013 (KR) 10-2013-0011175

(51) Int. Cl. A61B 5/0408; A61B 5/00

(52) U.S. Cl. CPC A61B 5/0408; A61B 5/6891
USPC 600/395; 600/372

(57) ABSTRACT

The electrode structure for measuring a bio-signal and an apparatus for measuring an electrocardiogram using the same are disclosed. The electrode structure for measuring a bio-signal, comprises an electrode pad to measure bio-signals based on capacitive coupling between the electrode pad and human skin; an absorption layer formed at one side of the electrode pad; and a preamplifier which is electrically connected with the electrode pad and filters noise from the bio-signals from the electrode pad and amplifies and outputs the signals. Thus, the initial noise stabilization time can be reduced, and stable electrocardiogram signals can be obtained quickly without noises.

### CROSS-REFERENCE TO RELATED APPLICATION

This application claims priority to Korean Patent Application No. 10-2013-0011175, filed on Jan. 31, 2013, and all the benefits accruing therefrom under 35 U.S.C. §119, the contents of which in its entirety are herein incorporated by reference.

### BACKGROUND

1. Field

The present invention relates to an electrode structure for measuring a bio-signal and an apparatus for measuring an electrocardiogram using the same, and in particular to an electrode structure and an apparatus for measuring an electrocardiogram using the same which make it possible to obtain quickly a stable electrocardiogram signal while reducing noise stabilization time at an earlier stage without producing any noises.

2. Description of the Related Art

Various electric bio-signals such as an ECG (Electrocardiogram) signal, a brain signal, an electrocardiogram signal, etc. are generally measured from a human body and is used for the sake of health care.

The ECG (Electrocardiogram) is a kind of action current on the basis of an expansion and contraction of heart muscles. The action electric potential generating when heart muscles contract and expand serves to generate currents which spread from a heart to whole body, and it is known that a corresponding current generates a potential difference depending on the position of a human body. The above-mentioned potential difference can be measured and recorded by way of an electrode which is attached to an outer portion of a human body. The measured and recorded bio-signal is electrocardiogram.

A basic method to measure an electrocardiogram signal is a “12-lead” measuring method which is conducted in such a way that an electrode for a direct sensing is attached to a human body, and an electric activity of a heart is measured by way of the attached electrode.

In the above-mentioned measuring method, six electrodes are generally attached to a chest near a heart, and an electrode is attached to an arm and a leg, respectively, so the electrocardiogram is measured by the attached 10 electrodes. Afterwards, the voltage obtained from each electrode is added or subtracted, thus producing twelve signals consisting of “v1-v6, I-III, aVR, aVL, aVF”.

Since the above-mentioned measuring method requires the electrodes directly attached to a human body, it has problems such as a longer inspection time, an inconvenience caused to a testee and a disposal of wasted skin-contact electrodes.

In order to improve the above-mentioned problems, there is newly used a new measuring method called an electric non-contact electrocardiogram measuring method which has features in that an electrocardiogram can be indirectly measured in a state that a testee dos not take off his clothes without a direct human body contact of electrodes.

The electric non-contact electrocardiogram measuring method has features in that an electrocardiogram can be measured with the aid of capacitance formed between a human body skin and electrodes in a state that a testee does not take off his clothes. For example, the Korean Registered Patent Number 10-0736721 discloses an electric non-contact apparatus and method for taking electrocardiograms, which can be adapted to a seat for a vehicle.

Meanwhile, it is important that an electrocardiogram should be accurately measured without producing any noises.

In case of the electric non-contact electrocardiogram measuring method, a signal measurement can be stably performed in a couple seconds or a couple minutes because the electric non-contact electrocardiogram measuring method may be influenced by humidity, textiles of testee's clothes and its thicknesses before the measurement starts after the testee sits in the chair. The problem is that it requires a longer noise stabilization time until a clear signal without noises is obtained.

### SUMMARY

Accordingly, it is an object of the present invention to provide an electrode structure for measuring a bio-signal and an apparatus for measuring an electrocardiogram using the same which make it possible to obtain quickly a stable electrocardiogram signal in such a way to reduce noise stabilization time at an earlier stage without producing any noises.

The technical problems to be improved by the present invention are not limited to the above-mentioned problems, and the other problems not mentioned in the above might be clearly understood by those who skilled in the art along with the following descriptions.

To achieve the above object, there is provided an electrode structure for measuring a bio-signal, comprising an electrode pad measuring bio-signals on the basis of capacitance at a skin of a human body; an absorption layer formed at one side of the electrode pad; and a preamplifier which is electrically connected with the electrode pad and filters noise from the bio-signals from the electrode pad and amplifies and outputs the signals.

The absorption layer is formed of a superabsorbent polymer.

The superabsorbent polymer of the absorption layer is one selected among a polymer produced by graft-polymerizing acrylonitrile to starch or cellulose, a block copolymer of acrylic acid and vinyl alcohol, and a block copolymer of salt and vinyl alcohol.

The absorption layer is a multiple-layer structure formed of an upper absorption layer, an intermediate absorption layer and a lower absorption layer which are sequentially stacked on from one side of the electrode pad.

The intermediate absorption layer is formed of a superabsorbent polymer so as to absorb moisture in the air, and the upper absorption layer and the lower absorption layer serve to reinforce and protect the intermediate absorption layer and are formed of a vapor permeability fiber for the purpose of allowing the moisture to penetrate from the surrounding air into the intermediate absorption layer.

The absorption layer is formed at a side facing a skin of a human body between both sides of the electrode pad.

The electrode pad is formed of an electrically conductive fiber, and the electrode pad is formed of a metallic electrode.

To achieve the above objects, there is provided an apparatus for measuring electrocardiogram using an electrode structure for measuring a bio-signal, comprising a first electrode and a second electrode measuring bio-signals; a differential measurement amplifier for differentially amplifying the output signals of the first electrode and the second electrode as they are inputted into the differential measurement amplifier; a signal process unit for receiving an output signal of the differential measurement amplifier and filtering noises and amplifying the filtered signals; and a wireless sensor node for analog-digital converting the output signal of the signal process unit and transmitting wirelessly, and each of the first electrode and the second electrode comprising an electrode pad connected on the basis of capacitance at a skin of a human body; an absorption layer formed at one side of the electrode pad; and a preamplifier which is electrically connected with the electrode pad and noise-filters the bio-signals from the electrode pad and amplifies and outputs the signals.

Each of the absorption layers of the first electrode and the second electrode is formed of a superabsorbent polymer.

There is further provided an earthed circuit for eliminating the common mode noises of the first electrode and the second electrode.

At one side of the earthed electrode belonging to the earthed circuit is provided an absorption layer formed of a superabsorbent polymer.

When the electrocardiogram measuring apparatus is installed at a chair, the first electrode and the second electrode are spaced apart from each other and are installed at the backrest of the chair, and the earthed electrode is installed at a sitting part of the chair.

### Advantageous Effects of the Invention

According to the electrode structure for measuring a bio-signal and the apparatus for measuring an electrocardiogram using the same of the present invention, it is possible to obtain quickly a stable electrocardiogram signal in such a way to reduce noise stabilization time at an earlier stage without producing any noises.

In addition, according to the electrode structure for measuring a bio-signal and the apparatus for measuring an electrocardiogram using the same of the present invention, it is possible to obtain quickly a stable electrocardiogram signal in such a way to lower the possibility of static electricity in an environment with low humidity in the air due to seasonal or geographical conditions.

### BRIEF DESCRIPTION OF THE DRAWINGS

The above and other aspects, features and advantages of the disclosed exemplary embodiments will be more apparent from the following detailed description taken in conjunction with the accompanying drawings in which:

FIG. 1 is a view illustrating an electrode structure according to the present invention;

FIG. 2 is a view illustrating a construction of an electrode structure according to an embodiment of the present invention to which the principle of FIG. 1 is substantially adapted;

FIG. 3 is a view illustrating a construction of an electrocardiogram measuring apparatus according to an embodiment of the present invention; and

FIG. 4 is a view illustrating an example of use of the electrocardiogram measuring apparatus of FIG. 3.

### DETAILED DESCRIPTION

The electrode structure for measuring a bio-signal and an apparatus for measuring an electrocardiogram using the same according to a preferred embodiment of the present invention will be described with reference to the accompanying drawings.

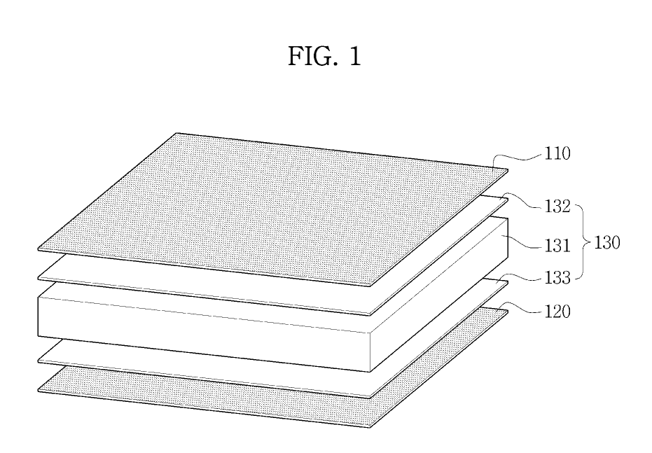

FIG. 1 is a view illustrating an electrode structure according to the present invention.

It seems that the long noise stabilization time taking place at an earlier stage in the conventional electric non-contact electrocardiogram measuring method is caused due to static electricity (which mainly resides in the electrodes or testee's clothes).

Meanwhile, it is known that the static electricity is directly related with humidity and the kinds of clothes. The higher the humidity, the fewer the occurrence of humidity. For example, in the winter when humidity is relatively higher, static electricity tends to occur more frequently as compared to the summer. In the summer when humidity is higher than 60% RH, static electricity dies out along with the moisture in the air. In the winter when humidity is lower than 30-40% RH, static electricity resides in the air because it still lingers in the air.

The average humidity in Asia countries, North American countries, European countries is about 40-60HR, in which environment it is very hard to obtain electrocardiogram signal quickly without noises in the electric non-contact electrocardiogram measuring method. In addition, in case that noises are high due to static electricity, the measurement of electrocardiogram signal may be impossible.

In terms of the electric non-contact electrocardiogram measuring method, if a testee sits in a chair with attached electrodes in a state that electrodes or part of a testee's body contain moisture, the static electricity does not matter, so the noise stabilization time at earlier stage may be reduced in an attempt to obtain electrocardiogram signals.

The present invention suggests a capacitance-measuring type electrode structure for measuring a bio-signal and an apparatus for measuring an electrocardiogram using the same which are invented on the basis of the above-mentioned features.

As shown in FIG. 1, the electrode structure is configured in a sandwich structure consisting of two opposite electrode pads 110 and 120 and a moisture absorption layer 130 disposed between the two opposite electrode pads 110 and 120.

Here, each of the electrode pads 110 and 120 is made from an electrically conductive fiber or metallic electrodes for the sake of electric conductions.

For example, the electrically conductive fiber may be formed of a copper-coated or nickel-coated polyester filament. The metallic electrode may be formed of a metallic substance such as copper, an alloy of copper and platinum or an alloy of copper and gold.

In an embodiment of the present invention, the moisture absorption layer 130 disposed between two electrode pads 110 and 120 may be made from superabsorbent polymer.

The superabsorbent polymer is a polymer resin produced by adapting a bridge coupling or an insoluble matter to a polymer electrolyte and can absorb water a couple hundred times the weight of itself and can keep it.

The above-mentioned superabsorbent polymer is substantially used in the form of powder or fiber states (paper diaper, sanitary pad, etc). It can be prepared by polymerizing molecules with functional groups which can easily ionize like —OH or —COOH or can easily hydrogen couple with water.

The above-mentioned moisture absorption layer 130 consists of superabsorbent polymer such as polymer produced by graft-polymerizing acrylonitrile to starch or cellulose, block copolymer of acrylic acid and vinyl alcohol, and block copolymer of salt and vinyl alcohol.

The absorption layer 130 formed of superabsorbent polymer serves as a moisture absorption sponge. When it is disposed between the two electrode pads 110 and 120 which are formed in a sandwich structure, it absorbs moistures from the surrounding air and helps reduce electric charges accumulated in the electrode pads 110 and 120, so it is possible to measure stably and quickly electrocardiogram signals without producing any noises.

Here, the absorption layer 130 may be formed in a single-layer structure or in a multiple-layer structure.

The absorption layer 130 formed in a multiple-layer structure, as shown in FIG. 1, comprises an upper absorption layer 132, an intermediate absorption layer 131 and a lower absorption layer 133, which are positioned in an order from one surface of the electrode pad 110.

Here, the intermediate absorption layer 131 may be formed of a superabsorbent polymer for the purpose of absorbing moistures in the air.

The upper absorption layer 132 and the lower absorption layer 133 serve to reinforce and protect the superabsorbent polymer which forms the intermediate absorption layer 131 while reinforcing the strength of the intermediate absorption layer 131 and preventing transformation. In addition, the upper absorption layer 132 and the lower absorption layer 133 are configured not to prevent the absorption of moisture to the intermediate absorption layer 131. For the sake of this, the upper absorption layer 132 and the lower absorption layer 133 may be made from fibers with moisture permeability.

The electrode pads 110 and 120 each are so equipped with an absorption layer 130 that the electrodes can keep moisture all the time. In this state, when a testee sits in a chair equipped with the electrode pads 110 and 120, the electrocardiogram can be measured, by which it is possible to reduce the initial noises when obtaining electrocardiogram signals and to greatly reduce the initial noise stabilization time which is required so as to obtain clear signals.

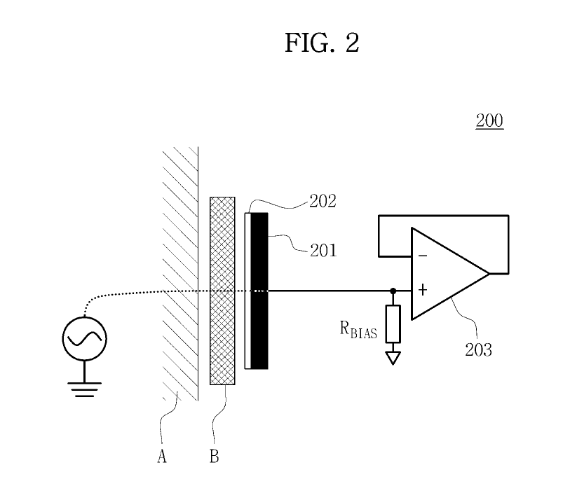

FIG. 2 is a view illustrating a construction of an electrode structure according to an embodiment of the present invention to which the principle of FIG. 1 is substantially adapted.

The electrode structure 200 according to an embodiment of the present invention is a capacitance electrode of a skin non-contact type for the sake of measuring bio-signals. The electrode pad 201 of the electrode structure 200 is capacitive-coupled with a skin A of a testee's human body when measuring electrocardiogram signals with an electrically non-conductive substance layer B such as clothes, blanket, leather, etc. being disposed between them.

More specifically, the electrode structure 200 comprises an electrode pad 201 designed to obtain a bio-signal of a testee based on a capacitance formation at a human body skin A, an absorption layer 202 formed on one surface of the electrode pad 201 and minimizing the effects of the static electricity, and a preamplifier 203 electrically connected with the electrode pad 201 for filtering noises from the bio-signals inputted through the electrode pad 201 and amplifying the same. A bias resistor R_{BIAS} is connected between the non-inverted (+) terminal of the preamplifier 203 and the ground.

An absorption layer 202 is provided at one side of the electrode pad 201 for the sake of static electricity prevention. The absorption layer 202 corresponds to the absorption layer 130 as shown in FIG. 1. It may be formed in a single layer structure or a multiple-layer structure. According to an embodiment of the present invention, the absorption layer 202 may comprise a superabsorbent polymer and may be formed at a surface facing the skin surface A of a human body between both surfaces of the electrode pad 201.

The electrode structure 200 comes to induce an electrocardiogram signal from the skin of a testee with the aid of a human body skin A of a testee, an electrically non-conductive substance layer B like clothes, an electrically conductive fiber or an electrode pad 201 which is a metallic member.

Here, the skin A of the testee serves as an electrode side facing the electrode pad 201, and the electrically non-conductive substance layer B like clothes serves as an insulator, by which a capacitance-based measurement can be implemented. The skin A, the electrically non-conductive substance layer B and the electrode pad 201 all serve as the capacitor.

Since the value of the capacitance C affects the construction of the circuit of the preamplifier 203 connected to the output terminal in the course of capacitance formation, the value of the capacitance C can be computed by the following formula 1 for the sake of circuit designs,

$$C = {ɛ}_{0} {ɛ}_{r} \frac{A}{d} \qquad [ Formula 1 ]$$

In the formula 1, the values of the capacitance are determined by the parameters of the dielectric constant (∈₀), the non-dielectric constant (∈ᵣ), the thickness (d) of the electrically non-conductive substance layer B and the size A of the electrode pad 201.

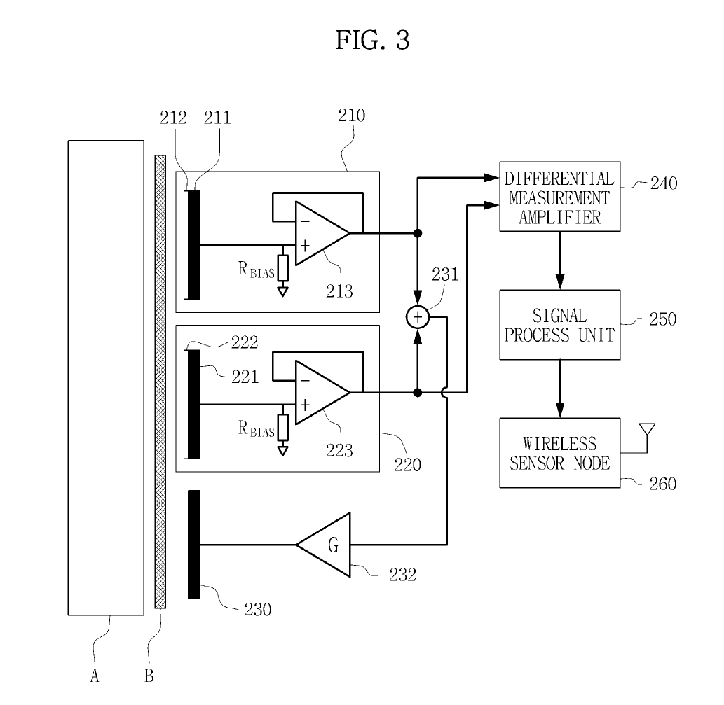

FIG. 3 is a view illustrating a construction of an electrocardiogram signal measuring apparatus according to an embodiment of the present invention.

As shown in FIG. 3, the electrocardiogram signal measuring apparatus according to an embodiment of the present invention comprises a first electrode 201 and a second electrode 220 for measuring the bio-signals of a human body, a differential measurement amplifier 240 for receiving the output signals of the first electrode 210 and the second electrode 220 for thereby differentially amplifying the signals, a signal process unit 250 electrically connected to the output terminal of the differential measurement amplifier 240, and a wireless sensor node 260 wirelessly transmitting digitalized signals of the signal process unit 250 to the external server, etc.

The first electrode 210 and the second electrode 220 are skin non-contact electrodes for the sake of measurement of bio-signals and are same as the construction of the electrode structure as described in FIG. 2.

The differential measurement amplifier 240 serves to amplify the potential difference between the first electrode 210 and the second electrode 220 and outputs to the signal process unit 250.

The signal process unit 250 receives an output signal of the differential measurement amplifier 240 and filters noises and amplifies the filtered signals and obtain an electrocardiogram signal.

The wireless sensor node 260 serves to perform an analog-digital conversion with respect to the output signal from the signal process unit 250 and to transmit wirelessly to the external server, etc. for thereby performing the monitoring operations of display, process, storage, etc. of the electrocardiogram signals.

In more details, the first electrode 210 comprises an electrode pad 211 connected with the skin A of the human body on the basis of capacitance with the electrically non-conductive substance layer B such as clothes being disposed between them, an absorption layer 212 formed on one side of the electrode pad 211, and a preamplifier 213 which filters the noises from the bio-signals from the electrode pad 211 and amplifies and outputs the same.

In the same manner, the second electrode 220 comprises an electrode pad 221 spaced apart from and facing the skin A, an absorption layer 222 formed on one side of the same, and a preamplifier 223.

Each electrode 210, 220 comes to amplify an electrocardiogram signal which is a small bio-signal inputted through a displacement current without a direct body contact and convert into voltages and output to the differential measurement amplifier 240. At this time, at one side of each of the electrodes 210 and 220 are provided absorption layers 212 and 222 for thereby measuring bio-signals stably and quickly while preventing the occurrence of static electricity.

According to an embodiment of the present invention, there may be further provided an earthed circuit for the purpose of eliminating common mode noises of the first electrode 210 and the second electrode 220. The earthed circuit comprises an adder 231, an amplifier 232 and an earthed electrode 230.

The thusly constituted earthed circuit has features in that the output signals of the first electrode 210 and the second electrode 220 are summed through the adder 231, and the common mode noise components are eliminated, and the output signal of the adder 231 is amplified by the amplifier 232 with a negative gain and is fed back to the human body through the earthed electrode 230. The electric potential of the human body is forced to drop in the opposite way as the common mode noises for thereby lowering the level of the common mode noises. According to the embodiment of the present invention, an absorption layer with a superabsorbent absorption polymer may be provided at one side of the earthed electrode 230 for the purpose of enhancing the prevention effects of static electricity.

In the electrocardiogram signal measuring apparatus, the electrocardiogram signals which are bio-signals can be induced by two electrode pads 211 and 221 from the skin A of the testee, and the electrocardiogram signals induced by the electrode pads 211 and 221 by the preamplifiers 213 and 223 serving as the high pass filter are noise-filtered and amplified. The electrocardiogram signals processed through the preamplifiers 213 and 223 are transferred to the earthed circuit. The earthed circuit serves to extract the common mode components from the bio-signals inputted from the two electrodes 210 and 220 and invert the same and amplify and apply to the human body through the earthed electrode 230, so it is possible to reduce the common mode noises in the course of measurement of electrocardiogram signals. As the gain of the earthed electrode 230 increases, the common mode noises decrease more.

The differential measurement amplifier 240 is adapted to amplify the potential difference between the electrocardiogram signals induced by the two electrodes 211 and 221. The output signals from the differential measurement amplifier 240 passes through the signal process unit 250 and are noise-filtered, so the electrocardiogram signals are obtained.

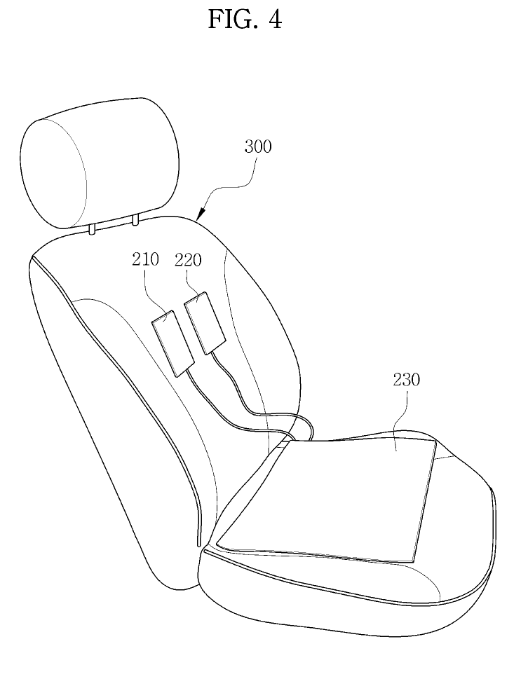

FIG. 4 is a view illustrating a use example of the electrocardiogram measuring apparatus of FIG. 3.

As shown in FIG. 4, when the electrocardiogram signal measuring apparatus according to an embodiment of the present invention is adapted to the seat 300 of a vehicle, etc., the first electrode 210 and the second electrode 220 are spaced-apart and installed at the backrest of the seat 300 against which a testee's back or both shoulders are supported, and the earthed electrode 203 is installed over the seating portions of the seat 300 in which the testee sits.

The absorption layers 212 and 222 are provided at the first electrode 210 and the second electrode 220, respectively, so the occurrence of the static electricity can be prevented, and the noise stabilization time at the earlier stage can be reduced, and at the moment the testee sits in the chair, the electrocardiogram of the testee can be measured in stable and quick ways without noises.

It is obvious that the electrode structure for measuring a bio-signal and an apparatus for measuring an electrocardiogram using the same according to the present invention may be changed or modified in various forms within the ranges the technical concepts of the present invention allows.

What is claimed is:

1. An electrode structure for measuring a bio-signal, comprising:
an electrode pad to measure bio-signals based on capacitive coupling between the electrode pad and human skin;
an absorption layer formed at one side of the electrode pad; and
a preamplifier to filter noise of the bio-signals obtained from the electrode pad, amplifiy and output the signals, wherein the preamplifier is electrically connected with the electrode pad.

2. The structure of claim 1, wherein the absorption layer comprises a superabsorbent polymer.

3. The structure of claim 1, wherein the superabsorbent polymer of the absorption layer is one selected among a polymer produced by graft-polymerizing acrylonitrile to starch or cellulose, a block copolymer of acrylic acid and vinyl alcohol, and a block copolymer of salt and vinyl alcohol.

4. The structure of claim 1, wherein the absorption layer is a multiple-layer structure comprising an upper absorption layer, an intermediate absorption layer and a lower absorption layer which are sequentially stacked on from one side of the electrode pad.

5. The structure of claim 1, wherein the intermediate absorption layer is formed of a superabsorbent polymer so as to absorb moisture in the air, and the upper absorption layer and the lower absorption layer serve to reinforce and protect the intermediate absorption layer and are formed of a vapor permeability fiber for the purpose of allowing the moisture to penetrate from the surrounding air into the intermediate absorption layer.

6. The structure of claim 1, wherein the absorption layer is formed at a side faced the human skin of both sides of the electrode pad.

7. The structure of claim 1, wherein the electrode pad is formed of an electroconductive fiber.

8. The structure of claim 1, wherein the electrode pad is formed of a metallic electrode.

9. An apparatus for measuring electrocardiogram using an electrode structure for measuring a bio-signal, comprising:
a first electrode and a second electrode to measure bio-signals;
a differential measurement amplifier for differentially amplifying the output signals of the first electrode and the second electrode as they are inputted into the differential measurement amplifier;
a signal process unit to receive an output signal of the differential measurement amplifier, filter noises and amplify the filtered signals; and
a wireless sensor node for analog-digital converting the output signal of the signal process unit and transmitting wirelessly, and
wherein each of the first electrode and the second electrode comprising:
an electrode pad which is capacitive coupled with human skin;
an absorption layer formed at one side of the electrode pad; and
a preamplifier which is electrically connected with the electrode pad and noise-filters the bio-signals from the electrode pad and amplifies and outputs the signals.

10. The apparatus of claim 9, wherein each of the absorption layers of the first electrode and the second electrode comprises a superabsorbent polymer.

11. The apparatus of claim 9, further comprising:
an earthed circuit to eliminate the common mode noises of the first electrode and the second electrode.

12. The apparatus of claim 11, wherein at one side of the earthed electrode belonging to the earthed circuit is provided an absorption layer comprising a superabsorbent polymer.

13. The apparatus of claim 11, wherein when the electrocardiogram measuring apparatus is installed at a chair, the first electrode and the second electrode are spaced apart from each other and are installed at the backrest of the chair, and the earthed electrode is installed at a sitting part of the chair.
